(19) United States
(12) Patent Application Publication
(10) Pub. No.: US 2018/0140547 A1
(43) Pub. Date: May 24, 2018

(54) COMPOSITIONS AND METHODS FOR ADMINISTRATION OF AN ENZYME TO A SUBJECT'S AIRWAY

(71) Applicant: BOARD OF REGENTS, THE UNIVERSITY OF TEXAS SYSTEM, Austin, TX (US)

(72) Inventors: Robert O. WILLIAMS, III, Austin, TX (US); Steven IDELL, Tyler, TX (US)

(21) Appl. No.: 15/571,529

(22) PCT Filed: May 6, 2016
(86) PCT No.: PCT/US16/31087

§ 371 Date: Nov. 3, 2017

Related U.S. Application Data
(60) Provisional application No. 62/157,839, filed on May 6, 2015.

(51) Int. Cl. A61K 9/00; C12N 9/72; A61P 11/00

(52) U.S. Cl. CPC A61K 9/0078; C12N 9/6459; A61P 11/00; C12Y 304/21068; C12Y 304/21073; C12N 9/6462

(57) ABSTRACT

Methods and composition for delivery of enzymes to a subject's airway. In some aspects, nebulized composition of enzymes, such as plasminogen activators are provided. In further aspects perfluorocarbon compositions comprising enzymes, such as plasminogen activators are provided. Compositions may, in some aspects, be used for the treatment of lung infections or acute lung injury, such as inhalational smoke induced acute lung injury (ISALI).

The present application claims the priority benefit of U.S. provisional application No. 62/157,839, filed May 6, 2015, the entire contents of which are incorporated herein by reference.

### BACKGROUND OF THE INVENTION

### 1. Field of the Invention

The present invention relates generally to the field of molecular biology, drug delivery and medicine. More particularly, it concerns compositions and methods for the delivery of therapeutic enzymes compositions to a subject's respiratory system.

### 2. Description of Related Art

Many patients with severe burns and smoke exposure develop a severe form of ALI (Acute Lung Injury) called the Acute Respiratory Distress Syndrome (ARDS) that is associated with a mortality of 30-40%, protracted hospitalization and long-term morbidity.

Inhalational smoke (IS)-induced ALI (ISALI) is characterized by severe airway obstruction, fibrinous airway casts or debris and alveolar fibrin deposition. Effective, specific treatment for ISALI is now lacking.

Burn injuries affect over 1 million patients in the United Sates annually and ISALI affects thousands of smoke-exposed patients in civilian and military practice annually (Enkhbaatar et al., 2004a). ISALI contributes to more than 3000 deaths and 17,000 fire-related injuries in the United States annually and a fire-related mortality rate of 2-3/100,000 population, which is one of the highest in the developed world [Committee on injury and poison prevention (2000) Pediatrics 105:1355-1357]. Supportive care is suboptimal, protracted and expensive. Outcomes entail significant mortality and morbidity. Despite current supportive care including mechanical ventilation, the mortality rate of ARDS, including that associated with ISALI, approaches 30-40 percent (Phua et al., 2009).

These considerations demand the testing of new and potentially more effective therapy. ISALI is associated with severe respiratory impairment, protracted hospitalization and, often, the requirement for mechanical ventilation. Long-term complications of ISALI include bronchial reactivity, accelerated pulmonary fibrosis and bronchiectasis. Among all forms of ALI, ISALI is especially prone to aberrant fibrin turnover including fibrin casts that form in the large airways and fibrin in the alveoli (Enkhbaatar et al., 2004a). Bronchial casts interfere with gas exchange, often require bronchoscopic clearance and promote atelectasis. While nebulized heparin and N-acetylcycteine are used in clinical practice, the efficacy of nebulized heparin in patients with ISALI remains unproven in any randomized or prospectively controlled clinical trials (Tuinman et al., 2012), nor has heparin alone been shown to improve ISALI when tested in our studies in sheep (Enkhbaatar et al., 2008a; Enkhbaatar et al., 2008b). Heparin does not clear established clots and nebulized heparin can initiate systemic coagulopathy in ISALI (O'Donnell, 2012).

### SUMMARY OF THE INVENTION

Provided herein is method of preparing an enzyme solution for administration to a subject's airway that includes nebulizing the enzyme solution (e.g., using a vibrating mesh nebulizer). The enzyme can be a tissue plasminogen activator, which includes a single chain urokinase plasminogen activator (scuPA) and a tissue plasminogen activator (tPA). In some embodiments, the vibrating mesh nebulizer is an AERONEB® Professional Nebulizer or an EZ Breathe Atomizer.

Thus, in a first embodiment there is provided a method of preparing an enzyme solution for administration to a subject's airway comprising nebulizing the enzyme solution to provide a nebulized solution. In certain aspects, the enzyme may be a plasminogen activator, such as a single chain urokinase plasminogen activator (scuPA) or a tissue plasminogen activator (tPA). In certain aspects, nebulizing the enzyme solution may be by using a vibrating mesh nebulizer. In some aspects, nebulizing the enzyme solution does not comprise use of a jet nebulizer or an ultrasonic nebulizer. In certain aspects, nebulizing an enzyme solution of the embodiments may comprise providing sufficient nebulization energy and/or time to provide a nebulized solution having a median droplet size of between about 2.5 μm and 10 μm, 2.5 μm and 8 μm, or 3.0 μm and 6 μm. In some specific aspects, nebulizing the enzyme solution comprises obtaining a lyophilized enzyme composition, reconstituting the lyophilized enzyme composition in a solution (e.g., an aqueous solution) to provide an enzyme solution, and nebulizing the enzyme solution. Thus, in a further embodiment, there is provided a nebulized enzyme solution produced in accordance with the methods of the embodiments.

In still a further embodiment, there is provided a composition comprising a nebulized solution of scuPA or tPA. In some aspects, composition or enzyme solution of the embodiments may be an aqueous solution. In certain aspects, the enzyme solution comprises a physiologically acceptable salt concentration and/or a pH buffering agent. For example, enzyme solution may be a sterile saline solution or phosphate buffered saline (PBS). In preferred aspects, the composition or enzyme solution comprises scuPA.

In a further embodiment, a method treating a subject is provided comprising administering a nebulized enzyme solution (e.g., a tPA and/or scuPA enzyme solution) to the airway of a subject in need thereof. For example, the subject may have an acute lung injury or infection. In still further aspects, subject for treatment has inhalational smoke induced acute lung injury (ISALI), chemical-induced lung injury, plastic bronchitis, severe asthma, or acute respiratory distress syndrome (ARDS). In this embodiment, the plasminogen activator is nebulized using nebulizer, such as a vibrating mesh nebulizer (e.g., the AERONEB® Professional Nebulizer or the EZ Breathe Atomizer). The skilled artisan understands that any type of atomizer, such as a nebulizer, that delivers a therapeutically and pharmaceutically acceptable dose of the enzyme is suitable for use according to the embodiments.

In a further embodiment, there is provided a composition comprising a plasminogen activator and a perfluorocarbon (e.g., a “breathing liquid”). In some aspects, the plasminogen activator is scuPA and/or tPA. In some aspects, the perfluorocarbon may comprise a cycloalkyl group. For example, the perfluorocarbon may be perfluorodecalin and/or perfluoro-octylbromide.

Another further embodiment of the invention provides a method for treating a subject having a lung infection or lung injury comprising administering to the subject a therapeutically effective amount of a composition comprising a plasminogen activator and a perfluorocarbon. In some aspects, the plasminogen activator is a scuPA or a tPA. In certain aspects, the perfluorocarbon may be perfluorodecalin and/or perfluoro-octylbromide.

In certain aspects, a solution or composition of the embodiments, such as nebulized solution comprising an enzyme (e.g., uPA, scuPA or tPA) is essentially free of non-physiological surfactants. Non-physiological surfactants of the embodiments can comprise ionic surfactants or non-ionic surfactants. For example, the surfactants include block copolymer surfactants (e.g., block copolymers of propylene oxide and ethylene oxide, PLURONIC® surfactants, such as PLURONIC®-F68 surfactant) and polysorbates (e.g., TWEEN® surfactants, such as polysorbate 40 and/or polysorbate 80 (TWEEN®-80)). In certain aspects, a composition or solution of the embodiments (e.g., a nebulized composition comprising uPA, scuPA or tPA) is essentially free of a non-ionic surfactant, such as polysorbate 80 and/or F-68 surfactant (PLURONIC® F68).

In still further aspects, a solution or composition of the embodiments comprises a surfactant, such as a non-physiological surfactant. For example, in some aspects a nebulized composition of the embodiments comprises uPA or scuPA and at least a first surfactant. For example, in some aspects, a nebulized composition comprises a non-ionic surfactant such as polysorbate 20 (PS20), polysorbate 40 (PS40) or polysorbate 80 (PS80). In further aspects, a nebulized solution comprises from about 0.01% to about 0.1% (e.g., between 0.01% and 0.07% or between 0.01% and 0.05%) of a non-ionic surfactant such as a polysorbate surfactant (e.g., PS40 or PS80). In yet further aspects, a nebulized solution of the embodiments comprises from about 0.01% to about 0.1% (e.g., between 0.01% and 0.07% or between 0.01% and 0.05%) of F-68 surfactant (CAS Number 9003-11-6).

In some aspects, the pharmaceutical composition of the embodiments, such as a nebulized enzyme solution, is essentially free of any impurities. The pharmaceutical composition may be essentially free of polyvinylpyrrolidone, polyvinylalcohol, polyacrylate, or polystyrene. In some aspects, the pharmaceutical composition is essentially free of any polymeric excipients. The pharmaceutical composition may, for example, be essentially free of poloxamers, polyethylene glycol, or polypropylene glycol. In some aspects, the pharmaceutical composition is essentially free of any surfactants. In further aspects, the pharmaceutical composition is free of other compounds beyond the excipient and the active pharmaceutical composition.

In aspects of the embodiments, concern a pharmaceutical composition, such a nebulized enzyme composition (e.g., a nebulized composition comprising uPA, scuPA or tPA), that is administered via inhalation. The therapeutically effective amount may be administered to the patient in one inhalation or in 2 or more inhalations. In some aspects, the therapeutically effective amount is administered in 2, 3, or 4 inhalations. In some aspects, the method comprises administering the therapeutically effective amount to the patient once a day. In other aspects, the method comprises administering the therapeutically effective amount to the patient two or more times a day.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%, preferably below 0.001%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing that intratracheal delivery of recombinant scuPA in mice with bleomycin-induced ALI increases BAL uPA activity.

FIG. 2 shows that treatment of a sheep with nebulized scuPA provided detectable uPA activity associated with human uPA antigen after scuPA treatment (Lane 3). uPA antigen and activity were likewise found in lung homogenates (Lane 4) from the scuPA-treated animal. Lane 1: uPA standard and Lane 2: baseline uPA activity.

FIG. 3 is a schematic showing the methods of preparing several different nebulized single chain urokinase plasminogen activator (scuPA) formulations.

FIG. 4 is a schematic showing the activity of the nebulized scuPA formulations prepared according to FIG. 3.

FIG. 5 is a schematic showing the activity of tissue plasminogen activator (tPA) once mixed with perfluorodecalin (PFD) or perfluoro-octylbromide (PFB).

FIG. 6 is a schematic showing a comparison of the enzymatic activity of tPA.

FIG. 7 is an SDS PAGE gel showing the results of the nebulized tPA samples.

FIG. 8 is a schematic showing a comparison of enzymatic activity of uPA.

FIG. 9 shows an SDS PAGE gel of the results of the scuPA samples.

### DESCRIPTION OF ILLUSTRATIVE EMBODIMENTS

### I. The Present Embodiments

Provided herein is a method of preparing an enzyme solution for administration to a subject's airway comprising nebulizing the enzyme solution using a nebulizer, such as a vibrating mesh nebulizer. It is a surprising finding of the present studies detailed herein that nebulization of enzymes, such by using a vibrating mesh nebulizer, results in nebulized compositions that maintain significant enzymatic activity levels. Also provided herein is a method of treating lung injuries and infections, such as inhalational smoke induced acute lung injury (ISALI) in a subject by administering to the subject a therapeutically effective amount of a nebulized plasminogen activator via an airway. In some cases, the plasminogen activator is nebulized using a vibrating mesh nebulizer. Further provided herein is a composition comprising a plasminogen activator and a perfluorocarbon, and a method for using the plasminogen activator/perfluorocarbon composition to treat lung injury and infection (e.g., ISALI). In some embodiments, an enzyme for use according to the embodiments is a proenzyme. In further aspects, the enzyme a plasminogen activator.

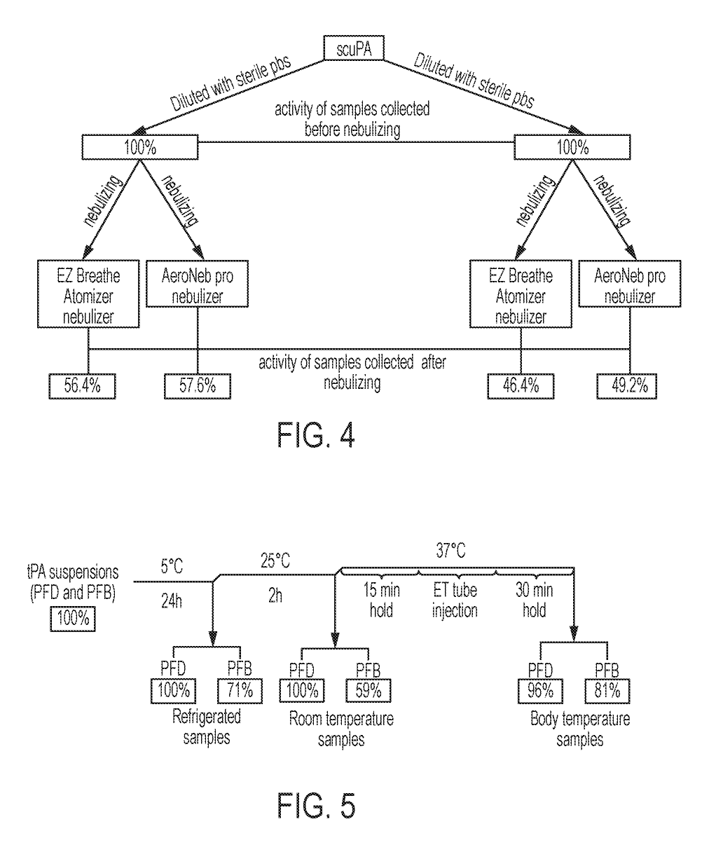

In still further embodiments, an enzyme for use herein is a plasminogen activator selected from tPA and scuPA. The terms “tissue plasminogen activator” and “tPA” are used interchangeably and refer herein to a serine protease (in some embodiments, EC 3.4.21.68) that can be involved in the conversion of plasminogen to plasmin. It should be understood that the terms “tissue plasminogen activator” and “tPA” include recombinant forms including, but not limited to, altepase, reteplase, tenecteplase, and desmoteplase. The terms “tissue plasminogen activator” and “tPA” further include the single chain form (sc-tPA), the two chain form (ds-tPA), and mixtures thereof In some embodiments, the tPA is a human tPA or a human-derived tPA. The terms “single chain urokinase plasminogen activator” and “scuPA” are used interchangeably and refer herein to a proenzyme of a urokinase serine protease (in some embodiments, EC 3.4.21.73), which serine protease can be involved in the conversion of plasminogen to plasmin. The “single chain urokinase plasminogen activator” or “scuPA” can be activated by proteolytic cleavage between Lys158 and Ile159, resulting in two chains linked by a disulfide bond that form the serine protease enzyme. Example 3 and FIG. 4 below describe the nebulization of scuPA using a vibrating mesh nebulizer and the surprisingly high enzymatic activity achieved following nebulization as compared to prior art methods of nebulization. In some embodiments, the vibrating mesh nebulizer is an AERONEB® Professional Nebulizer or an EZ Breathe Atomizer.

The term “enzyme solution” refers herein to any liquid formulation containing an enzyme that is suitable for nebulization. In some embodiments, the enzyme solution contains a pharmaceutically acceptable carrier or excipient as defined herein. The enzyme solution is administered to a subject's airway via inhalation or any other method known to those of skill in the art. The term “airway” refers herein to any portion of the respiratory tract including the upper respiratory tract, the respiratory airway, and the lungs. The upper respiratory tract includes the nose and nasal passages, mouth, and throat. The respiratory airway includes the larynx, trachea, bronchi and bronchioles. The lungs include the respiratory bronchioles, alveolar ducts, alveolar sacs and alveoli.

Also provided herein is a method of treating inhalational smoke induced acute lung injury (ISALI) in a subject comprising administering to the subject a therapeutically effective amount of a nebulized plasminogen activator via an airway, wherein the plasminogen activator is nebulized using a vibrating mesh nebulizer. In some embodiments, the plasminogen activator is selected from a tPA and a scuPA. In other or further embodiments, the vibrating mesh nebulizer is an AERONEB® Professional Nebulizer or an EZ Breathe Atomizer.

It should be understood that “treating ISALI” indicates partially or completely delaying, alleviating, mitigating or reducing the intensity of one or more attendant symptoms of a disorder or condition such as an ISALI condition and/or alleviating, mitigating or impeding one or more causes of a disorder or condition such as an ISALI condition. Treatments according to the invention may be applied preventively, prophylactically, pallatively or remedially. In some instances, the terms “treat,” “treating,” “treatment” and grammatical variations thereof include partially or completely reducing a condition or symptom associated with an ISALI condition as compared with prior to treatment of the subject or as compared with the incidence of such condition or symptom in a general or study population. In some embodiments, an ISALI condition includes one or more of: reduced oxygenation, airway obstruction (including a severe airway obstruction), fibrinous airway casts or debris, and alveolar fibrin deposition. Accordingly, treating an ISALI condition includes one or more of improvement of oxygenation, reduced airway obstruction, reduced fibrinous airway casts or debris, and reduced alveolar fibrin deposition. In some embodiments, an ISALI condition is treated with a reduced incidence of bleeding.

Further provided herein is a composition comprising a plasminogen activator and a perfluorocarbon (PFC). In some embodiments, the plasminogen activator in the composition is selected from a tPA and a scuPA. In other or further embodiments, the PFC in the composition is selected from perfluorodecalin, perfluoro-1,3-dimethylcyclohexane, FC-75, perfluorooctane and perfluoro-octylbromide. In some aspects, PFC is or comprises a PFC having a cycloalkyl group, such as perfluorodecalin, perfluoro-1,3-dimethylcyclohexane or FC-75. It should be understood that the plasminogen activator and PFC can be in any ratio or concentration. In some embodiments, the composition comprises a plasminogen activator at a concentration of approximately 0.005-0.040 mg/mL of PFC.

Still further provided is a method of treating inhalational smoke induced acute lung injury (ISALI) in a subject comprising administering to the subject a therapeutically effective amount of a composition comprising a plasminogen activator and a PFC. Example 4 and FIG. 5 demonstrate that a plasminogen activator, tPA, retained activity in a perfluorocarbon mixture. Further, the PFC and plasminogen activator additively foster airway debris removal as well as clearance of alveolar fibrin and improved outcome. Specifically, the PFC effectively delivers the plasminogen activator which promotes 1) dissolution and dislodgement of the airway casts; and 2) removal of airway and alveolar debris while supporting respiratory gas exchange. Mechanistically, the PFC effectively recruits lung volume. Given the low surface tension of the PFC liquid, the PFC distributes the plasminogen activator throughout the lung, potentially between casts and airway wall, thus breaking down the casts as they are being formed while slowing formation of new casts. As the PFC volatizes from the lung, the plasminogen activator remains to further act to dissolve airway casts and alveolar fibrin. Upon redosing with PFC suspensions, the PFC volumes not only deposit additional drug but dislodge the casts and alveolar debris. Because the PFC is incompressible, it stents open damaged small airways and thereby aids recruitment. Although we are not bound by any certain mechanism, contact with PFCs may also protect the underlying epithelium through attenuation of coagulation, which is initiated by tissue factor in the small airways and alveoli in virtually all forms of ALI. With in-line suctioning, the lower density debris float in the relatively more dense PFC, facilitating removal of airway fibrin cast fragments and debris.

Accordingly, in some embodiments of the method of administering a plasminogen activator and PFC composition, the plasminogen activator is selected from a tPA and a scuPA. In other or further embodiments of the method of administering a plasminogen activator and PFC composition, the PFC in the composition is selected from perfluorodecalin and perfluoro-octylbromide.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. All patents, patent applications, and publications referenced herein are incorporated by reference in their entirety for all purposes. Term definitions used in the specification and claims are as follows.

### II. Pharmaceutical Compositions

It is contemplated that enzyme of the embodiments, such as uPA, scuPA and/or tPA, can be administered for the treatment of damaged lung tissue and/or to prevention damage to lung tissues. In preferred aspects, the polypeptides are delivered locally to the airway, such as administration of a nebulized formulation. They can be administered alone or in combination with anti-fibrotic compounds.

It is not intended that the present invention be limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects.

Such compositions are typically prepared as liquid solutions or suspensions. Suitable diluents and excipients are, for example, water, saline, dextrose, glycerol, or the like, and combinations thereof. In addition, if desired, the compositions may contain minor amounts of auxiliary substances, such as wetting or emulsifying agents, stabilizing agents, or pH buffering agents.

Generally, pharmaceutical compositions may comprise an effective amount of one or more of the enzymes of the embodiments or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition that contains at least one polypeptide of the embodiments isolated by the method disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier is incompatible with the active ingredient, its use in the pharmaceutical compositions is contemplated.

Further in accordance with certain aspects of the embodiments, the composition suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a composition contained therein, its use in administrable composition for use in practicing the methods is appropriate. Examples of carriers or diluents include fats, oils, water, saline solutions, lipids, liposomes, resins, binders, fillers, and the like, or combinations thereof. The composition may also comprise various antioxidants to retard oxidation of one or more component. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

The pharmaceutical compositions described herein may comprise one or more excipients. Excipients are components which are not therapeutically active but may be used in the formation of a pharmaceutical composition. The excipients used herein include amino acids, sugars, sugar derivatives, or other excipients know those of skill in the art. In particular, the present disclosure includes the use of a sugar such as trehalose, lactose, glucose, fructose, or mannose, or a sugar derivative such as an aminosugar such as glucosamine or a sugar alcohol such as mannitol. Other excipients which may be used include amino acids such as alanine or glycine.

In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

In a specific embodiment, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates or lyoprotectants, such as dextrose, mannose, galactose, fructose, lactose, sucrose, maltose, sorbitol, mannitol, etc.

In further aspects of the embodiments may concern the use of a pharmaceutical lipid vehicle composition that includes enzymes (e.g., uPA, scuPA or tPA) of the embodiments, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the polypeptides of the embodiments may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active enzyme. In other embodiments, an active enzyme may comprise between about 2% to about 75% of the weight of the unit, or between about 25% to about 60%, for example, and any range derivable therein. Naturally, the amount of active enzyme in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In some aspects, a pharmaceutical formulation comprises, or is essentially free of, one or more surfactant. Surfactants used in accordance with the disclosed methods include ionic and non-ionic surfactants. Representative non-ionic surfactants include polysorbates, such as PS-20, PS-40 (TWEEN®-20) and PS-80 (TWEEN-80®) surfactants (ICI Americas Inc. of Bridgewater, N.J.); poloxamers (e.g., poloxamer 188); TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or(e.g., lauroamidopropyl); myristamidopropyl-, palmidopropyl-, or isostearamidopropyl-dimethylamine; sodium methyl cocoyl-, or disodium methyl oleyl-taurate; MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. For example, the surfactant can be present in a formulation in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). However, in further aspects, a pharmaceutical formulation of the embodiments is essentially free of non-ionic surfactants or essentially free of all surfactants.

In some aspects, a surfactant can be added to a pre-lyophilized enzyme, to a lyophilized enzyme, or to a enzyme that is reconstituted in aqueous or non-aqueous solvent. Enzymes and surfactants in the solid phase can be combined using co-grinding techniques, as known in the art. See e.g., Williams et al. (1999) Eur J Pharm Biopharm 48:131-40.

As detailed above in some aspects a composition of the embodiments comprises or is essentially free of a non-physiological surfactant. The term “non-physiological” is used herein to describe a quality of not being found in a mammalian subject. Thus, non-physiological surfactants of the embodiments exclude surfactant lipids obtained from a mammalian subject, for example SURVANTA® surfactant (Abbott Laboratories Corp. of Abbott Park, Ill.), ALVEOFACT® surfactant (Boehringer Ingelheim of Ingelheim, Germany), and similar physiological surfactants. See e.g., Gunther et al. (2001) Respir Res 2:353-64 and references cited therein. Non-physiological surfactants also exclude recombinantly produced or synthesized surfactants that are normally found in a mammalian subject.

A composition of the embodiments can also comprise additional agents for protein stabilization, including other surfactants. Thus, a formulation of the invention can comprise a combination of surfactants. A formulation can also comprise sucrose to enhance protein stability and retard aggregation. See e.g., Kim et al. (2001) J Biol Chem 276:1626-33.

Further provided herein is a composition comprising an enzyme of the embodiments (e.g., uPA, scuPA or tPA) and a perfluorocarbon (PFC). In some aspects, the PFC in the composition is selected from perfluorodecalin, perfluoro-1,3-dimethylcyclohexane, FC-75, perfluorooctane and perfluoro-octylbromide. In some aspects, PFC is or comprises a PFC having a cycloalkyl group, such as perfluorodecalin, perfluoro-1,3-dimethylcyclohexane or FC-75. It should be understood that the plasminogen activator and PFC can be in any ratio or concentration. In some embodiments, the composition comprises a plasminogen activator at a concentration of approximately 0.005-0.040 mg/mL of PFC.

Still further provided is a method of treating lung injury or disease (e.g., inhalational smoke induced acute lung injury (ISALI)) in a subject comprising administering to the subject a therapeutically effective amount of a composition comprising an ezyme of the embodiments (e.g., uPA, scuPA or tPA) and a PFC. Accordingly, in some embodiments a method of administering an enzyme and PFC composition is provided, wherein the PFC in the composition is selected from perfluorodecalin and perfluoro-octylbromide.

### III. Aerosol Dispersion and Nebulizing Devices

The formulations of the embodiments can be aerosolized using any suitable device, including but not limited to a jet nebulizer, an ultrasonic nebulizer, a metered dose inhaler (MDI), and a device for aerosolization of liquids by forced passage through a jet or nozzle (e.g., AERX® drug delivery devices by Aradigm of Hayward, Calif.). For delivery of a formulation to a subject, as described further herein below, an pulmonary delivery device can also include a ventilator, optionally in combination with a mask, mouthpiece, mist inhalation apparatus, and/or a platform that guides users to inhale correctly and automatically deliver the drug at the right time in the breath. Representative aerosolization devices that can be used in accordance with the methods of the present invention include but are not limited to those described in U.S. Pat. Nos. 6,357,671; 6,354,516; 6,241,159; 6,044,841; 6,041,776; 6,016,974; 5,823,179; 5,797,389; 5,660,166; 5,355,872; 5,284,133; and 5,277,175 and U.S. Published Patent Application Nos. 20020020412 and 20020020409.

Using a jet nebulizer, compressed gas from a compressor or hospital air line is passed through a narrow constriction known as a jet. This creates an area of low pressure, and liquid medication from a reservoir is drawn up through a feed tube and fragmented into droplets by the air stream. Only the smallest drops leave the nebulizer directly, while the majority impact on baffles and walls and are returned to the reservoir. Consequently, the time required to perform jet nebulization varies according to the volume of the composition to be nebulized, among other factors, and such time can readily be adjusted by one of skill in the art.

A metered dose inhalator (MDI) can be used to deliver a composition of the invention in a more concentrated form than typically delivered using a nebulizer. For optimal effect, MDI delivery systems require proper administration technique, which includes coordinated actuation of aerosol delivery with inhalation, a slow inhalation of about 0.5-0.75 liters per second, a deep breath approaching inspiratory capacity inhalation, and at least 4 seconds of breath holding. Pulmonary delivery using a MDI is convenient and suitable when the treatment benefits from a relatively short treatment time and low cost. Optionally, a formulation can be heated to about 25° C. to about 90° C. during nebulization to promote effective droplet formation and subsequent delivery. See e.g., U.S. Pat. No. 5,299,566.

Aerosol compositions of the embodiments comprise droplets of the composition that are a suitable size for efficient delivery within the lung. In some cases, a surfactant formulation is delivered to lung bronchi, more preferably to bronchioles, still more preferably to alveolar ducts, and still more preferably to alveoli. Aerosol droplets are typically less than about 15 μm in diameter, less than about 10 μm in diameter, less than about 5 μm in diameter, or less than about 2 μm in diameter. For efficient delivery to alveolar bronchi of a human subject, an aerosol composition may preferably comprises droplets having a diameter of about 1 μm to about 5 μm.

Droplet size can be assessed using techniques known in the art, for example cascade, impaction, laser diffraction, and optical patternation. See McLean et al. (2000) Anal Chem 72:4796-804, Fults et al. (1991) J Pharm Pharmacol 43:726-8, and Vecellio None et al. (2001) J Aerosol Med 14:107-14.

Protein stability following aerosolization can be assessed using known techniques in the art, including size exclusion chromatography; electrophoretic techniques; spectroscopic techniques such as UV spectroscopy and circular dichroism spectroscopy, and protein activity (measured in vitro or in vivo). To perform in vitro assays of protein stability, an aerosol composition can be collected and then distilled or absorbed onto a filter. To perform in vivo assays, or for pulmonary administration of a composition to a subject, a device for aerosolization is adapted for inhalation by the subject. For example, protein stability can be assessed by determining the level of protein aggregation. Preferably, an aerosol composition of the invention is substantially free of protein aggregates. The presence of soluble aggregates can be determined qualitatively using DLS (DynaPro-801TC, ProteinSolutions Inc. of Charlottesville, Va.) and/or by UV spectrophotometry.

The term “vibrating mesh nebulizer” refers herein to any nebulizer that operates on the general principle of using a vibrating mesh or plate with multiple aperatures (an aperture plate) to generate a fine-particle, low-velocity aerosol. Some nebulizers may contain a mesh/membrane with between 1000 and 7000 holes, which mesh/membrane vibrates at the top of a liquid reservoir (see, e.g., U.S. Patent Publn. 20090134235 and

Waldrep and Dhand 2008, each incorporated herein by reference). In some embodiments, the vibrating mesh nebulizer is an AERONEB® Professional Nebulizer, Omron MICROAIR®, Pari EFLOW® or an EZ Breathe Atomizer. In some aspects, a vibrating mesh nebulizer has a vibrating frequency of between about 50-250 kHz, 75-200 kHz 100-150 kHz or about 120 kHz. These devices have a high efficiency of delivering aerosol to the lung and the volume of liquid remaining in these devices is minimal, which is an advantage for expensive and potent compounds like plasminogen activators.

In certain aspects, a nebulized composition of the embodiments is produced using a vibrating mesh nebulizer. For example, the composition can be produced with an active vibrating mesh nebulizer (e.g., an Aeroneb® Professional Nebulizer System).

Descriptions of such system and there operation can be found, for instance, in U.S. Pat. Nos. 6,921,020; 6,926,208; 6,968,840; 6,978,941; 7,040,549; 7,083,112; 7,104,463; and 7,360,536, each of which is incorporated herein by reference in its entirety. In yet further aspects, a composition of the embodiments can be produced with a passive vibrating mesh nebulizer, such as the Omron MicroAir® or the EZ Breathe Atomizer.

### IV. Definitions

As used in the specification and claims, the singular form “a,” “an,” and “the” include plural references unless the context clearly dictates otherwise. For example, the term “a cell” includes a plurality of cells, including mixtures thereof.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “administering” refers to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. In some embodiments, the administration is via inhalation of a nebulized composition.

The term “airway” refers herein to any portion of the respiratory tract including the upper respiratory tract, the respiratory airway, and the lungs. The upper respiratory tract includes the nose and nasal passages, mouth, and throat. The respiratory airway includes the larynx, trachea, bronchi and bronchioles. The lungs include the respiratory bronchioles, alveolar ducts, alveolar sacs and alveoli.

A “composition” is intended to mean a combination of active agent and another compound or composition, inert (for example, a detectable agent or label) or active, such as an adjuvant.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.” “Mammal” for purposes of treatment refers to any animal classified as a mammal, including a human, domestic and farm animals, nonhuman primates, and zoo, sports, or pet animals, such as dogs, horses, cats, cows, etc.

The term “enzyme” refers herein to one or more polypeptides that catalyze a specific biochemical reaction or to a proenzyme. The term “proenzyme” refers to a biologically active substance that is metabolized into an enzyme. In one embodiment, the enzyme is a tissue plasminogen activator (tPA). In other or further embodiments, the enzyme is a proenzyme and is a single chain urokinase plasminogen activator (scuPA).

The term “fibrinolysin” refers herein to any of several proteolytic enzymes that promote the dissolution of blood clots. A fibrinolysin includes, but is not limited to, plasmin, tissue plasminogen activator (tPA, sc-tPA and dc-tPA), urokinase (uPA), and urokinase proenzymes (scuPA).

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software.

The terms “inhalational smoke induced acute lung injury” and “ISALI” are used interchangeably herein and refer to a form of acute lung injury (ALI) caused by smoke inhalation. ALI is also referred to as “mild ARDS.” ALI can be defined by finding one or more of the following conditions in a subject: 1) bilateral pulmonary infiltrates on chest x-ray, 2) when measured by right heart catheterization as clinically indicated, pulmonary capillary wedge pressure <18 mmHg (2.4 kPa), and 3) PaO2/FiO2<300 mmHg (40 kPa). In some embodiments, treatment of ISALI includes treatment of one or more of the following conditions: reduced oxygenation, airway obstruction (including a severe airway obstruction), fibrinous airway casts or debris, and alveolar fibrin deposition.

The terms “nebulizing,” “nebulized” and other grammatical variations, refer herein to the process of converting a liquid into small aerosol droplets. In some embodiments, the aerosol droplets have a median diameter of approximately 2-10 μm. In some embodiments, the aerosol droplets have a median diameter of approximately 2-4 μm.

The terms “perfluorocarbon” and “PFC” are used interchangeably and refer herein to an organofluorine compound that contains predominantly carbon and fluorine. It should be understood that the term “perfluorocarbon” is meant to include highly fluorinated molecules that contain molecules in addition to carbon and fluorine, and are commonly referred to as fluorocarbons. Examples of perfluorocarbons include, but are not limited to, perfluorodecalin, perfluoro-octylbromide, FC 77, PF 5060 and Rimar 101. PFCs used according to the present invention share similar physicochemical properties with respect to gas solubility, density and surface tension but may differ with respect to radio-opacity and kinematic viscosity which could have an impact on visualization and mobility of airway casts during debridement. Each listed perfluorocarbon includes all relevant isomers such as stereoisomers, enantiomers, and diastereomers.

The term “plasminogen activator” refers to a serine protease polypeptide that conversts plasminogen to plasmin, and includes, but is not limited to, tPA, uPA (two chain or active forms) and a proenzyme scuPA as defined herein.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” or “excipient” encompasses any of the standard pharmaceutical carriers, such as a phosphate buffered saline solution, saline (including sterile saline), water, and emulsions, such as an oil/water or water/oil emulsion, where “oil” represents the water immiscible phase of the emulsion that is pharmaceutically acceptable, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

The term “pharmaceutically acceptable salts” refers to any acid or base addition salt whose counter-ions are non-toxic to the subject to which they are administered in pharmaceutical doses of the salts. Specific examples of pharmaceutically acceptable salts are known to those of ordinary skill in the art.

The terms “pharmaceutically effective amount,” “therapeutically effective amount,” or “therapeutically effective dose” refer to the amount of a compound such as an

ACPD composition that will elicit the biological or medical response of a tissue, system, animal, or human that is being sought by the researcher, veterinarian, medical doctor or other clinician.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

The terms “prevent,” “preventing,” “prevention,” and grammatical variations thereof as used herein refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human.

The terms “pharmaceutically effective amount,” “therapeutically effective amount,” or “therapeutically effective dose” refer to the amount of a compound such as a tPA and/or scuPA composition that will elicit the biological or medical response of a tissue, system, animal, or human that is being sought by the researcher, veterinarian, medical doctor or other clinician.

The terms “single chain urokinase plasminogen activator” and “scuPA” are used interchangeably and refer herein to a proenzyme of a urokinase serine protease polypeptide (in some embodiments, EC 3.4.21.73), which serine protease can be involved in the conversion of plasminogen to plasmin, or to a proenzyme as described in U.S. Pat. No. 7,332,469, incorporated herein by reference. The “single chain urokinase plasminogen activator” or “scuPA” can be activated by proteolytic cleavage between Lys158 and Ile159, resulting in two chains linked by a disulfide bond that form the serine protease enzyme. It should be understood that scuPA homologs are also included in the present invention. The term “scuPA homolog” refers herein to homologs, orthologs, and paralogs of the proenzyme of the urokinase serine protease polypeptide identified as EC 3.4.21.73 and other sequences having greater than 70% homology to the proenzyme of the urokinase serine protease polypeptide identified as EC 3.4.21.73, or to a proenzyme as described in U.S. Pat. No. 7,332,469.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

The term “therapeutically effective amount” includes that amount of a compound such as a tPA and/or scuPA composition that, when administered, is sufficient to prevent development of, or alleviate to some extent, one or more of the symptoms of an ISALI abnormality being treated. The therapeutically effective amount will vary depending on the compound such as a tPA and/or scuPA composition, the disorder or conditions and their severity, the route of administration, the time of administration, the rate of excretion, the drug combination, the judgment of the treating physician, the dosage form, and the age, weight, general health, sex and/or diet of the subject to be treated.

The terms “tissue plasminogen activator” and “tPA” are used interchangeably and refer herein to a serine protease (in some embodiments, EC 3.4.21.68) that can be involved in the conversion of plasminogen to plasmin. It should be understood that the terms “tissue plasminogen activator” and “tPA” include recombinant forms including, but not limited to, altepase, reteplase, tenecteplase, and desmoteplase. The terms “tissue plasminogen activator” and “tPA” further include the single chain form (sc-tPA), the two chain form (ds-tPA), and mixtures thereof. In some embodiments, the tPA is a human tPA or a human-derived tPA. It should also be understood that tPA homologs are also included in the present invention. The term “tPA homolog” refers to homologs, orthologs, and paralogs of the tissue plasminogen activator polypeptide identified as EC 3.4.21.68 and other sequences having greater than 70% homology to the tissue plasminogen activator polypeptide identified as EC 3.4.21.68. In some embodiments, the tPA is a single chain form such as the ALTEPASE™ form.

The terms “treat,” “treating,” “treatment” and grammatical variations thereof as used herein include partially or completely delaying, alleviating, mitigating or reducing the intensity of one or more attendant symptoms of a disorder or condition such as an ISALI condition and/or alleviating, mitigating or impeding one or more causes of a disorder or condition such as an ISALI condition. Treatments according to the invention may be applied preventively, prophylactically, pallatively or remedially. In some instances, the terms “treat,” “treating,” “treatment” and grammatical variations thereof include partially or completely reducing a condition or symptom associated with an ISALI condition as compared with prior to treatment of the subject or as compared with the incidence of such condition or symptom in a general or study population. In some embodiments, an ISALI condition includes one or more of: reduced oxygenation, airway obstruction (including a severe airway obstruction), fibrinous airway casts or debris, and alveolar fibrin deposition. In some embodiments, an ISALI condition is treated with a reduced incidence of bleeding.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Intratracheal Delivery of Recombinant scuPA in Mice with Bleomycin-Induced ALI Increases BAL uPA Activity

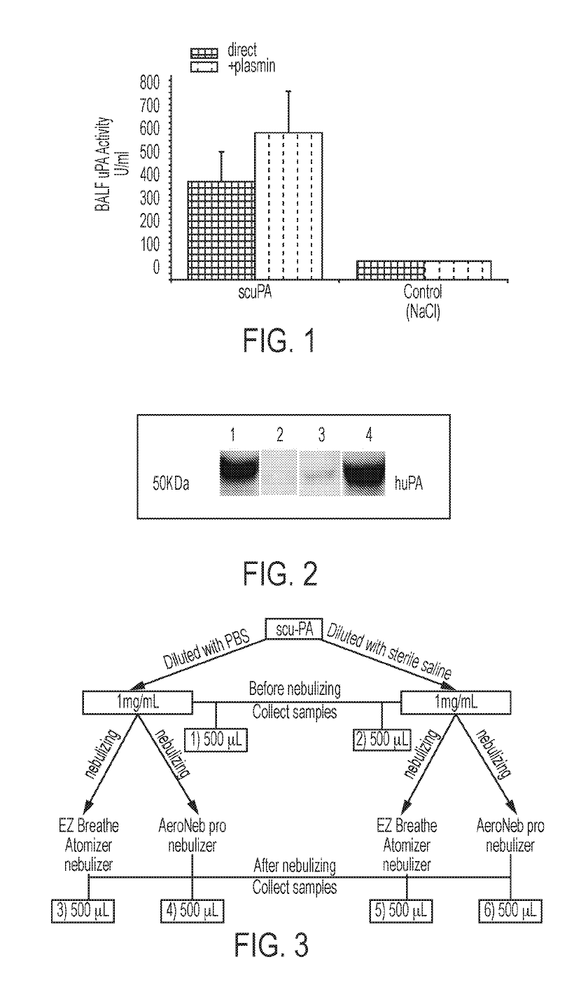

ALI was induced in C57/B6 mice with 2.5 U/kg bleomycin at day 0 (n=10 animals/group). Mice were treated daily with 25,000 U; 167 μg/mouse of recombinant human scuPA via a microsprayer (100 μl). At day 7, BAL was obtained 4 hours after the last microsprayer administration of scuPA. uPA activity was measured using the amidolytic substrate S-2444 (directly+after incubation with 1 mU/ml plasmin for 5 hours). The results are shown in FIG. 1. The increment of uPA activity after plasmin treatment suggested that some of the nebulized scuPA remained intact within the alveolar lining fluids and remained available for activation by plasmin in vivo. No pulmonary or systemic bleeding occurred. These data confirm those of a previous report showing that nebulized scuPA increases lavage fibrinolytic activity in trauma-induced AL and is well-tolerated (Munster et al., 2002). The findings also show that durable fibrinolytic activity of scuPA (a substantive increase 4 hours after aerosolization) was generated in the lungs in ALI.

### Example 2

### Nebulized scuPA Lyses Airway Casts and is Detectable in BAL of Sheep with ISALI

A sheep was treated with nebulized scuPA (2 mg/treatment begun 4 hours after induction of ISALI and continued every 4 hours×48 hours. Airway cast burden (obstruction score 12) fell into the range of sheep treated with nebulized tPA at 4 mg q 4 hours (vs. 20.7 in vehicle treated sheep with ISALI) (Enkhbaatar et al., 2004b). As shown in FIG. 2, BAL (bronchoalveolar lavage) of sheep had no detectable baseline uPA activity by fluorimetric analysis (Lane 2) but had detectable uPA activity associated with human uPA antigen after scuPA treatment (Lane 3) and uPA antigen and activity were likewise found in lung homogenates (Lane 4) from the scuPA-treated animal. Lane 1: uPA standard.

### Example 3

### Nebulized scuPA Lyses Airway Casts and is Detectable in BAL of Sheep with ISALI

Studies were also conducted on scuPA solutions containing 1 mg/mL of scuPA dissolved in either physiological buffered saline or normal saline, and then nebulized using two types of vibrating mesh nebulizers, the EZ Breathe Atomizer and the AeroNeb Pro nebulizer. scuPA readily dissolved in both liquid carriers. It was confirmed that the activity of scuPA before and after nebulization was not affected by the nebulizing conditions (e.g., solution formation, shear and temperature from the nebulizing process in the nebulizer). Also, it was confirmed that the median geometric particle size for the scuPA solutions was 3-4 microns with a narrow and acceptable size distribution. The materials, methods and results are provided below and a schematic of the procedure is provided in FIG. 3.

Phosphate Buffered Saline and Sterile Saline Preparation:

The phosphate buffered saline (DPBS, Lot 14190-250, Gibco) was purchased from Biostore at UT-Austin. The compositions of the PBS were as shown in Table 1.

TABLE 1

| | | | |
|---|---|---|---|
| | Molecular | Concentration | |
| Components | Weight | (mg/L) | mM |

Inorganic Salts

| | | | |
|---|---|---|---|
| Potassium Chloride (KCl) | 75 | 200 | 2.67 |
| Potassium Phosphate | 136 | 200 | 1.47 |
| monobasic (KH₂PO₄) | | | |
| Sodium Chloride (NaCl) | 58 | 8,000 | 137.93 |
| Sodium Phosphate dibasic | 268 | 2,160 | 8.06 |
| (Na₂HPO₄—7H₂O) | | | |

The pH of the PBS was 7.3±0.1. The sterile saline was purchased from B.Braun Medical Inc (Lot J1H573). Both preparations were stored at ambient room temperature, and excessive heat was avoided.

Device Introductions:

The EZ Breathe Atomizer nebulizer and AeroNeb pro nebulizer were used for testing. The Aeroneb® Professional Nebulizer System (vibrating mesh, Aerogen, Galway) was a portable medical device for multiple patient use. The Aeroneb® is intended to aerosolize physician-prescribed medications for inhalation that are approved for use with a general purpose nebulizer. The EZ Breathe Atomizer (vibrating mesh, Nephron Pharmaceuticals Corporation, USA) is a device that is intended to spray liquid medication in aerosol form into the air that a person will breathe. These devices can be used by patients with and without mechanical ventilation, or other positive pressure breathing assistance.

Scu-PA Solutions Preparation:

Eight vials each containing 3.5 mg/mL of scu-PA were obtained and stored at −80° C. In order to make two kinds of scu-PA solutions, the solutions were prepared in the following way:

A. scu-PA vials were held at room temperature (20° C.-25° C.) until they melted into solution. Two mL of the solution was transferred from the vial to a new 10 mL vial using a 6 mL syringe.

B. Using a 6 mL syringe with attached 21-gauge needle, 3 mL of sterile phosphate buffered saline (or sterile saline) was injected into one vial. The contents were manually agitated until all scu-PA solution was uniform. This solution was then diluted with sterile phosphate buffered saline (or sterile saline) to a final concentration of 1 mg/mL.

Geometric Particle-size Distribution (PSD) Testing:

Both nebulizers were loaded with the two kinds of 5 mL scu-PA at a concentration of 1 mg/mL as samples and pure saline and pure PBS as blank controls, separately (8 samples in total). The geometric particle-size distribution (PSD) was determined using a Malvern Spraytec. A standard nebulization procedure was performed 5 times; each test lasted for 5 seconds. All determinations were carried out at ambient room temperature, barometric pressure, and humidity.

Samples Collected from Nebulizer for Further Study:

After 10 seconds, the nebulized procedure was started, and run until the aerosol generation was stable, after which 500 μL samples of the nebulized output scu-PA (4 samples in total) were collected and then frozen in the −80° C. refrigerator. Six samples were prepared as shown in Table 2.

TABLE 2

| | With sterile phosphate | |
|---|---|---|
| | buffered saline | With sterile saline |
| Before nebulizing | 1(1) | 1(2) |
| After nebulizing | | |
| EZ Breathe Atomizer | 1(3) | 1(5) |
| nebulizer | | |
| AeroNeb pro nebulizer | 1(4) | 1(6) |

Total: 6

Results:

Table 3 denotes geometric particle-size distribution (PSD) information (n=5) of the samples and FIG. 4 shows the activity of each sample following nebulization.

TABLE 3

| Nebulizer | | | X(50%) | X(10%) | X(90%) |
|---|---|---|---|---|---|
| name | Sample | Solvent | μm | μm | μm |
| Aeroneb Pro | PBS | PBS | 3.14 | 0.87 | 7.74 |
| | Saline | Saline | 3.25 | 0.92 | 8.66 |
| | scu-PA | PBS | 3.83 | 0.96 | 11.40 |
| | scu-PA | Saline | 3.60 | 0.91 | 11.48 |
| EZ Breathe | PBS | PBS | 5.29 | 1.13 | 11.54 |
| Atomizer | Saline | Saline | 4.91 | 1.04 | 9.53 |
| | scu-PA | PBS | 4.79 | 1.16 | 11.17 |
| | scu-PA | Saline | 4.80 | 1.28 | 11.24 |

Based on these results, scuPA solutions are optimized for nebulization focusing on identifying the best nebulizer. Other parameters that are studied for nebulizer administration include confirming the effect of processing parameters (e.g., optimum solution composition for scuPA activity, aerodynamic properties including fine particle fraction, mass median aerodynamic diameter, and total emitted dose, temperature effects on scuPA activity during nebulization (e.g., using different nebulizer mechanism types), and the effects of shear (e.g., atomization pressure, ultrasonic vibrations, mesh size) of the liquid on scuPA activity.

### Example 4

### Suspension of tPA in PFCs is Stable and Preserves tPA Activity

FIG. 5 shows that suspension of tPA in PFCs is stable and that tPA activity is preserved. For these studies, the tPA/PFC suspensions (18 mL of the tPA-PFC suspension made at 0.22 mg/mL) were injected through the endotracheal tube (7 mm adult endotracheal tube) during the 50 minute hold time at 37° C. using a 10 mL syringe and 21 gauge needle. It was noted that the tPA was adequately wetted and deagglomerated in the PFCs. Other parameters that are studied for tPA administration include confirming the effect of processing parameters (e.g., particle size reduction of the tPA, viscosity of the PFC and resulting tPA-PFC suspension, solids content of the tPA-PFC suspension and its effect on administration during bronchoscopy on the tPA activity). The same approach is then used to analyze the formulations of the scuPA-PFC interventions.

The PFC and fibrinolysins additively foster airway debris removal as well as clearance of alveolar fibrin and improved outcome. Specifically, the PFC effectively delivers the fibrinolysins which promote 1) dissolution and dislodgement of the airway casts; and 2) removal of airway and alveolar debris while supporting respiratory gas exchange. Mechanistically, the PFC effectively recruits lung volume. Given the low surface tension of the PFC liquid, the PFC distributes the fibrinolysin throughout the lung, potentially between casts and airway wall, thus breaking down the casts as they are being formed while slowing formation of new casts. As the PFC volatizes from the lung, the fibrinolysin remains to further act to dissolve airway casts and alveolar fibrin. Upon redosing with PFC suspensions, the PFC volumes not only deposit additional drug but dislodge the casts and alveolar debris.

Because the PFC is incompressible, it stents open damaged small airways and thereby aids recruitment. Contact with PFCs may also protect the underlying epithelium through attenuation of coagulation, which is initiated by tissue factor in the small airways and alveoli in virtually all forms of ALI. With in-line suctioning, the lower density debris float in the relatively more dense PFC, facilitating removal of airway fibrin cast fragments and debris.

### Example 5

### Effect of Nebulization on tPA Activity

Nebulization of tPA Solution:

A commercial product, Cathflo® Activase®, was freshly reconstituted with DPBS (0.5 mg/mL). The solution was atomized using a vibrating mesh nebulizer. Two commercial brands of the vibrating mesh nebulizers; Aeroneb® Pro (Aerogen, Mountain View, Calif.) or EZ Breathe® Atomizer (Model EZ-100, Nephron Pharmaceuticals Corporation, Orlando, Fla.) were selected. Nebulization was stopped after there was no aerosol cloud observed. The condensate of each sample was collected in a polypropylene tube and kept at −20 ° C. until analyzed using human tPA activity ELISA kit (Molecular Innovations, Inc., Novi, Mich.). Enzyme activity of tPA before and after nebulization were calculated as percent recovery (with a coefficient of variation 10%).

SDS-PAGE Analysis:

Sodium dodecyl sulfate polyacrylamide gel electrophoresis (SDS-PAGE) based on the extraction method was used to determine physical loss of the protein and its degradation. tPA samples (6.5 μL and 7.5 μL for reduced and non-reduced samples, respectively) was mixed with 2.5 μL of concentrated (“4×”) NuPAGE® lithium dodecyl sulfate (LDS) sample buffer. One uL of NuPAGE® Reducing Agent (“10×”) was added to the reduced samples. All samples were heated at 90° C. for 2 min and then 10 μL of each samples loaded and separated on 4-12% NuPAGE® Novex® Bis-Tris gels. SDS running buffer was prepared by adding 50 mL of 20× NuPAGE® MES SDS running buffer to 950 mL of deionized water. The gel was run at 200 V for 35 min in MES SDS running buffer, and protein bands were visualized by SimplyBlue™ SafeStain (Thermo Fisher Scientific, Waltham, Mass.).

Effect of Nebulization on tPA Activity:

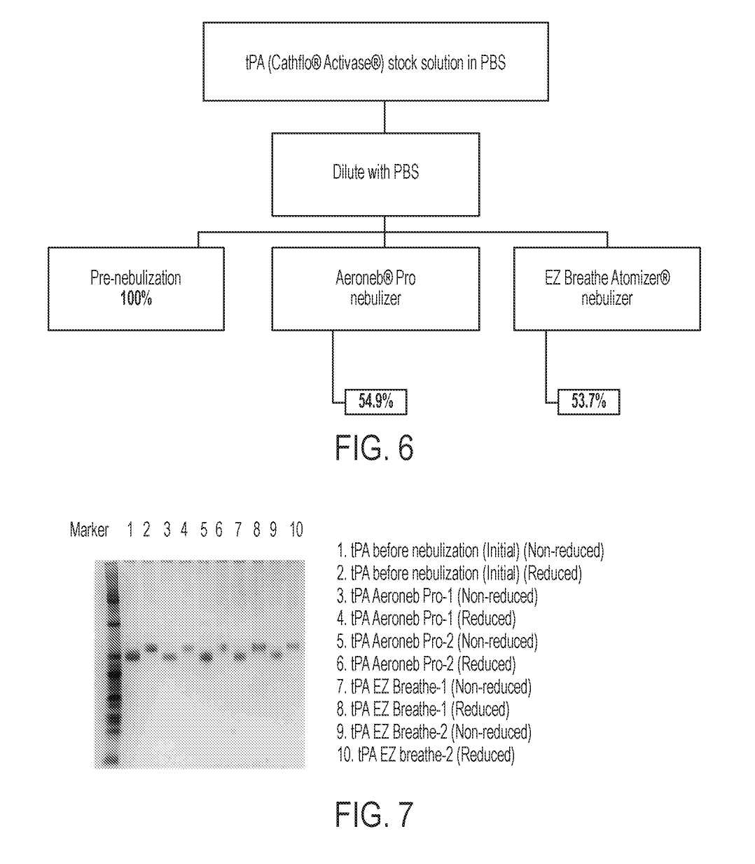

Lyophilized human tPA (Cathflow® Activase®) rapidly dissolved in DPBS. The results are shown in FIG. 6. Protein activity of tPA was not influenced by the nebulizing conditions (i.e. solution formation and temperature effect of the atomizing process of the nebulizers). The percentage of tPA activity from both vibrating mesh nebulizers was greater than 50% as compared to that of the solutions prior to nebulization. The Aeroneb®

Pro exhibited slightly better activity than EZ Breathe® nebulizer. Polyacrylamide gel electrophoresis was used to verify the tPA results. The loss of protein activity was associated with the physical loss of tPA. Thus, there was no significant change in the specific activity of protein after nebulization. Besides, the degradation products of tPA in all samples were not observed on the gels (FIG. 7).

### Example 6

### Nebulization of scuPA Solution

Formulation Preparation:

Lyophilized scuPA was prepared using a lyophilization process. In brief, a bulk solution of scuPA in DPBS containing 500 μg/mL of scuPA and 1.5% w/v of mannitol were prepared and sterile filtered using 0.2 μm SFCA sterile syringe filter (Corning Inc., Corning, N.Y.). Effect of filtration was also examined. Half milliliter of the filtrate was filled into a borosilicate glass vial and lyophilized using a VirTis Advantage Lyophilizer (VirTis Company Inc., Gardiner, N.Y.). Lyophilization cycle parameters were set as studied by Coldstream Laboratories, Inc., 2014 (Table 4). Primary drying time was varied by number of samples.

Nebulization of scuPA Solution:

Lyophilized scuPA was freshly reconstituted with sterile water for injection (250 μg/mL). Nebulization was demonstrated as described in Example 5. The solutions were atomized using two types of vibrating mesh nebulizers (Aeroneb® Pro and EZ Breathe® Atomizer). Each atomized sample was collected by condensation in the polypropylene tube and kept at −20 ° C. until tested. Human uPA activity ELISA kit and SDS-PAGE were used to analyze enzyme activity and the loss of the protein, respectively. Concentration of scuPA in the pre- and post-filtration and before and after nebulization were calculated as percent recovery of enzyme activity (with a coefficient of variation 10%).

SDS-PAGE Analysis:

SDS-PAGE was used to identify the loss of protein and its degradation products. The test method is as described in Example 5.

Effect of Sterile Filtration, Lyophilization and Nebulization on uPA Activity:

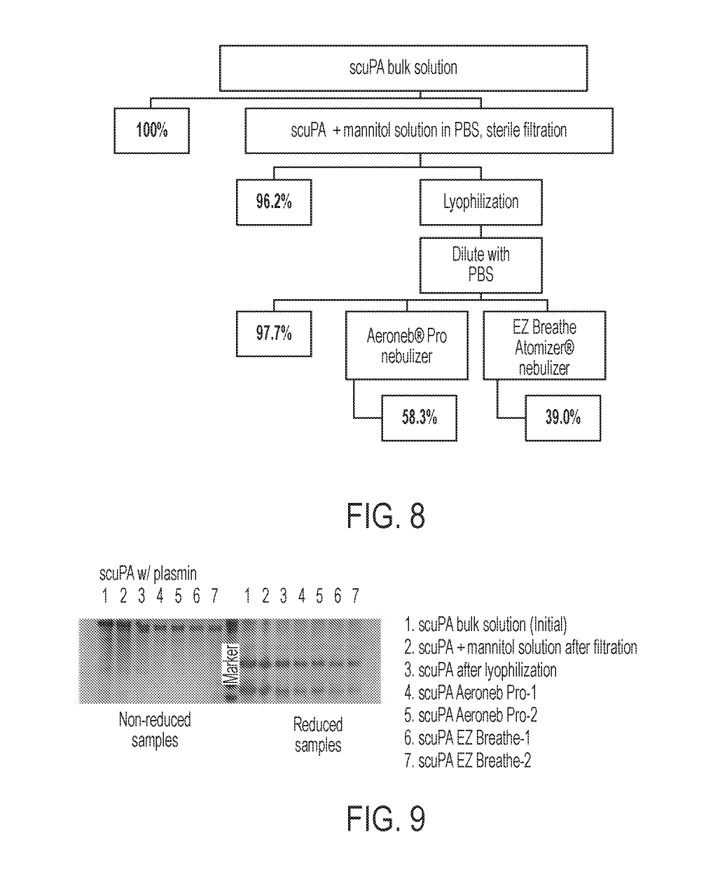

Lyophilized scuPA formulation was readily dissolved in water. FIG. 8 shows the protein activity of scuPA in the study. Neither sterile filtration, nor the lyophilization process affected the activity of scuPA. However, the activity of scuPA after atomization using the Aeroneb Pro® was almost 60%, while the other atomizer, EZ Breathe® provided about 40% activity. Regarding the SDS PAGE in FIG. 9, the loss of peptide activity was associated with the physical loss of the protein. Moreover, there were no degradation products detected in all samples.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Coldstream Laboratories, Inc. (2014). Formulation Development Report: Laboratory Scale Lyophilization Feasibility Study for scuPA for Injectio, 3.1 mg/vial.

Committee on injury and poison prevention (2000) Pediatrics 105:1355-1357

Enkhbaatar & Traber (2004) Clin.Sci.(Lond). 107:137-143

Enkhbaatar et al. (2004) Shock. 22:70-75

Enkhbaatar et al. (2008). Clin.Sci. (Lond). 114:321-329

Enkhbaatar, Herndon, and Traber (2008) J Burn Care Res. 30:159-162

O'Donnell (2012). J.Pharm.Pract 25:22-29

Munster et al. (2002) Blood Coagul. Fibrinolysis 13:591-601

Phua et al. (2009) Am J Respir Crit Care Med. 179:220-227

Tuinman et al. (2012). Crit Care 16:R70

Waldrep and Dhand, (2008) Current Drug Delivery, 5(2):114-119

U.S. Patent Publn. 20090134235

What is claimed is:

1. A method of preparing an enzyme solution for administration to a subject's airway comprising nebulizing the enzyme solution to provide a nebulized solution, wherein said solution is essentially free of non-physiological surfactants.

2. The method of claim 1, wherein said solution is essentially free of surfactants.

3. The method of claim 1, wherein the enzyme is a plasminogen activator.

4. The method of claim 3, wherein the plasminogen activator is a single chain urokinase plasminogen activator (scuPA) or a tissue plasminogen activator (tPA).

5. The method of claim 3, wherein the plasminogen activator is a scuPA.

6. The method of claim 1, wherein nebulizing the enzyme solution is by using a vibrating mesh nebulizer.

7. The method of claim 6, wherein the vibrating mesh nebulizer is an AERONEB® Professional Nebulizer or an EZ Breathe Atomizer.

8. The method of claim 6, wherein said nebulizing does not comprise use of a jet nebulizer or an ultrasonic nebulizer.

9. The method of claim 1, wherein the enzyme solution is an aqueous solution.

10. The method of claim 9, wherein the enzyme solution comprises a physiologically acceptable salt concentration.

11. The method of claim 9, wherein the enzyme solution comprises a pH buffering agent.

12. The method of claim 9, wherein the enzyme solution is phosphate buffered saline (PBS).

13. The method of claim 9, wherein the enzyme solution comprises scuPA.

14. The method of claim 1, wherein nebulizing the enzyme solution comprises:
(i) obtaining a lyophilized enzyme composition;
(ii) reconstituting the lyophilized enzyme composition in an aqueous solution to provide an enzyme solution; and
(iii) nebulizing the enzyme solution.

15. The method of claim 1, wherein nebulizing the enzyme solution comprises providing sufficient nebulization energy and/or time to provide a nebulized solution having a median droplet size of between about 2.5 μm and 10 μm.

16. The method of claim 15, wherein nebulizing the enzyme solution comprises providing sufficient nebulization energy and/or time to provide a nebulized solution having a median droplet size of between about 2.5 μm and 8 μm.

17. The method of claim 16, wherein nebulizing the enzyme solution comprises providing sufficient nebulization energy and/or time to provide a nebulized solution having a median droplet size of between about 3.0 μm and 6 μm.

18. A nebulized enzyme solution produced by a method according to any one of claims 1 to 17.

19. The method of any one of claims 1 to 17, further comprising administering the nebulized solution to the airway of a subject in need thereof

20. The method of claim 19, wherein the subject has an acute lung injury or infection.

21. The method of claim 20, further comprising administering the nebulized solution to the airway of a subject having a chemical-induced lung injury.

22. The method of claim 20, further comprising administering the nebulized solution to the airway of a subject having plastic bronchitis, asthma or acute respiratory distress syndrome (ARDS).

23. The method of claim 20, further comprising administering the nebulized solution to a subject having inhalational smoke induced acute lung injury (ISALI).

24. A method of treating inhalational smoke induced acute lung injury (ISALI) in a subject comprising administering to the subject a therapeutically effective amount of a nebulized plasminogen activator composition via an airway, wherein the nebulized plasminogen activator composition is essentially free of non-physiological surfactants.

25. The method of claim 24, wherein the plasminogen activator is nebulized using a vibrating mesh nebulizer.

26. The method of claim 24, wherein the nebulized plasminogen activator composition is essentially free of surfactants.

27. The method of claim 24, wherein the plasminogen activator is a single chain urokinase plasminogen activator (scuPA) or a tissue plasminogen activator (tPA).

28. The method of claim 24, wherein the plasminogen activator is a scuPA.

29. A composition comprising a nebulized solution of single chain urokinase plasminogen activator (scuPA) or a tissue plasminogen activator (tPA), wherein the nebulized solution is essentially free of non-physiological surfactants.

30. The composition of claim 29, wherein the solution is an aqueous solution.

31. The composition of claim 30, wherein the solution comprises a physiologically acceptable salt concentration.

32. The composition of claim 30, wherein the solution comprises a pH buffering agent.

33. The composition of claim 32, wherein the solution is phosphate buffered saline (PBS).

34. The composition of claim 29, comprising scuPA.

35. The composition of claim 29, wherein the nebulized solution has a median particle size of between about 2.5 μm and 10 μm.

36. The composition of claim 29, wherein the nebulized solution has a median particle size of between about 2.5 μm and 8 μm.

37. The composition of claim 29, wherein the nebulized solution has a median particle size of between about 3.0 μm and 6 μm.

38. A composition comprising a nebulized solution of single chain urokinase plasminogen activator (scuPA) and from about 0.01% to about 0.1% of a non-ionic surfactant.

39. The composition of claim 38, wherein comprising from about 0.01% to about 0.08% of a non-ionic surfactant.

40. The composition of claim 38, wherein the non-ionic surfactant comprises a polysorbate surfactant.

41. The composition of claim 38, wherein the non-ionic surfactant comprises F-68 surfactant.

42. The composition of claim 38, wherein the nebulized solution has a median particle size of between about 2.5 μm and 8 μm.

43. The composition of claim 42, wherein the nebulized solution has a median particle size of between about 3.0 μm and 6 μm.

44. A composition for use in the treatment of acute lung injury or lung infection, said composition comprising a nebulized solution in accordance with any one of claims 29-43.

45. The composition of claim 44, for use in the treatment of chemical-induced lung injury, plastic bronchitis, asthma, acute respiratory distress syndrome (ARDS) or inhalational smoke induced acute lung injury (ISALI).

46. A composition for use in the treatment of inhalational smoke induced acute lung injury (ISALI), said composition comprising a nebulized solution in accordance with any one of claims 29-43.

47. A composition comprising a plasminogen activator and a perfluorocarbon.

48. The composition of claim 47, wherein the composition is essentially free of non-physiological surfactants.

49. The composition of claim 48, wherein the composition is essentially free of surfactants.

50. The composition of claim 47, wherein the plasminogen activator is a single chain urokinase plasminogen activator (scuPA) or a tissue plasminogen activator (tPA).

51. The composition of claim 47, wherein the plasminogen activator is a scuPA.

52. The composition of claim 47, wherein the perfluorocarbon comprises a cycloalkyl group.

53. The composition of claim 47, wherein the perfluorocarbon is selected from perfluorodecalin and perfluoro-octylbromide.

54. The composition of claim 47, wherein the perfluorocarbon comprises perfluorodecalin.

55. A composition for use in the treatment of acute lung injury or infection infection, said composition comprising a composition in accordance with any one of claims 47-54.

56. The composition of claim 55, for use in the treatment of chemical-induced lung injury, plastic bronchitis, asthma, acute respiratory distress syndrome (ARDS) or inhalational smoke induced acute lung injury (ISALI).

57. A composition for use in the treatment of inhalational smoke induced acute lung injury (ISALI), said composition comprising a composition in accordance with any one of claims 47-54.

58. A method for treating a subject having a lung infection or lung injury, comprising administering an effective amount of a composition in accordance with any one of claims 47-54 to the airway of the subject.

59. The method of claim 58, wherein the subject has chemical-induced lung injury.

60. The method of claim 59, wherein the subject has plastic bronchitis, acute respiratory distress syndrome (ARDS) or inhalational smoke induced acute lung injury (ISALI).

61. A method for treating a subject having inhalational smoke induced acute lung injury (ISALI) comprising administering an effective amount of a composition in accordance with any one of claims 47-54 to the airway of the subject.

62. A method of treating inhalational smoke induced acute lung injury (ISALI) in a subject comprising administering to the subject a therapeutically effective amount of a composition comprising a plasminogen activator and a perfluorocarbon.

63. The method of claim 62, wherein the composition is essentially free of non-physiological surfactants.

64. The method of claim 63, wherein the composition is essentially free of surfactants.

65. The method of claim 62, wherein the plasminogen activator is a single chain urokinase plasminogen activator (scuPA) or a tissue plasminogen activator (tPA).

66. The method of claim 62, wherein the plasminogen activator is a scuPA.

67. The method of claim 62, wherein the perfluorocarbon is selected from perfluorodecalin and perfluoro-octylbromide.

68. The method of claim 67, wherein the perfluorocarbon comprises perfluorodecalin.
